(19) United States
(12) Patent Application Publication
(10) Pub. No.: US 2013/0324473 A1
(43) Pub. Date: Dec. 5, 2013

(54) Coagulating Collagen and Means for Preparing Same

(71) Applicant: Thomas Graeve, Stuttgart (DE)

(72) Inventor: Thomas Graeve, Stuttgart (DE)

(73) Assignee: AMEDRIX GmbH, Esslingen (DE)

(21) Appl. No.: 13/984,462

(22) PCT Filed: Jan. 26, 2012
(86) PCT No.: PCT/EP2012/000336

§ 371 Date: Aug. 8, 2013

(30) Foreign Application Priority Data
Feb. 9, 2011 (DE) 10 2011 011 092.5

(51) Int. Cl. A61K 38/39; A61M 5/24

(52) U.S. Cl. CPC A61K 38/39; A61M 5/2448
USPC 514/17.1; 530/356; 604/82

(57) ABSTRACT

The invention relates to the preparation and provision of a gelling collagen composition that instantaneously forms a collagen matrix, and means for the preparation and use thereof, in particular as part of a therapeutic treatment.

### FIELD OF INVENTION

The invention relates to the preparation and provision of a gelling collagen composition that instantaneously forms a collagen matrix, and means for the preparation and use thereof, in particular as part of a therapeutic treatment.

### BACKGROUND

In conjunction with degenerative or traumatic joint diseases in the human or animal body, cartilage defects occur in the form of missing, eroded cartilage material. Damaged articular cartilage has only limited capacity for self-regeneration. Treatment requires “filling in” the cartilage defect in order to replace the missing cartilage material. This is performed by means of invasive surgical procedures. A well-known treatment of cartilage defects is the transplantation of cartilage-forming, in particular autologous, cells such as chondrocytes into the defect for the purpose of triggering the formation of new hyaline cartilage (cartilage transplants) at that site. Other, in particular cell-free, methods employ certain materials and material compositions that can serve as artificial cartilage (cartilage implant). Known cartilage implants form a matrix or scaffold, into which cartilage cells of the surrounding intact cartilage tissue can migrate to thus form a new cartilaginous structure.

From DE 10 026 789 A1, a collagen-based biomatrix and methods for preparation thereof are known. A collagen biomatrix is prepared from collagen that has been extracted from rat tail tendons. For this purpose the acidic collagen solution that is present after the extraction is neutralized in the cold with serum-containing buffer and is cast, optionally in the presence of cells, to form a collagen gel that later gels or crosslinks, i.e. cures, to form a collagen-containing biomatrix that can be obtained in this manner as a ready-made cell-free collagen implant or cell-containing collagen transplant having embedded chondrocytes.

In known surgical procedures, the cartilage defects are first dissected, with any damaged cartilage tissue already being selectively removed in the process. This results in a cavity, which must then be selectively filled with a cartilage implant. Close contact between the cartilage implant and the surrounding tissue allows cartilage cells to infiltrate, thereby enabling the cartilage to regenerate. For this to occur, known cartilage implants, which are provided in ready-made form, must additionally be cut to size during surgery and fitted into the preparative cavity. In particular, the edges of the cartilage implant must engage the edges of the cavity in such a way that sturdy anchoring of the implant in the articular cartilage is achieved. Fitting the ready-made implant to the cavity is a complex and time-consuming process. As a result of these complex measures, the risk of contamination or of complications during surgery increases. Furthermore, it is not possible with these measures to achieve a complete fit with little surgical effort, and there is a risk of postoperative complications or of the treatment not being successful. Existing methods and means for the treatment of cartilage defects are therefore in need of improvement.

### SUMMARY OF INVENTION

The present invention to provides novel methods and means for preparing a collagen-based matrix that can be used for treating cartilage defects in the human or animal body and can allow the disadvantages known from the prior art to be avoided.

In one aspect, the invention provides a method whereby an instantaneously gelling collagen composition is obtained from a liquid concentrated collagen solution in combination with a liquid buffer solution. This collagen composition can be injected directly into the cavity of a cartilage defect, where it instantaneously cures to form a collagen matrix, which then forms the cartilage implant directly in situ. Placing the liquid gelling collagen composition into the cavity facilitates an improved fit to the shape of the cavity, and the still liquid collagen composition can come into direct contact with the edges and the bottom of the cavity.

### BRIEF DESCRIPTION OF DRAWINGS

FIG. 1 shows a schematic view of an embodiment of the filled syringe according to the invention as a means for carrying out the method according to the invention.

FIG. 2 shows the results of rheological studies on collagen gels: elastic modulus as a function of test frequency (mean values and standard deviations) in two-chamber syringes brought to different desired temperatures.

### DETAILED DESCRIPTION

According to the invention, the method comprises at least the following steps: concentrated liquid collagen solution and liquid buffer solution provided separately from each other that were in particular stored in the cold prior beforehand are jointly brought to a temperature ranging from 20° C. to about 37° C., preferably to about 30° C.; the collagen solution at the desired temperature and the buffer solution at the desired temperature are, preferably immediately thereafter, mixed with each other, whereby a gellable collagen composition is obtained that instantaneously begins to gel and is capable of curing to form a collagen biomatrix.

In a particular embodiment, the collagen solution and buffer solution are mixed only at the time of the application, i.e., in particular during the application of the collagen composition, and the initially still liquid gelling collagen composition is not created until the time of the application. According to one embodiment of the invention, the gelling but still liquid collagen composition therefore is applied in the nascent state, and it can then cure at the application site.

A concentrated collagen solution and a buffer solution are furnished which can be stored together unmixed but in an integral container, namely in a known manner in the cold, in particular at temperatures ranging from about 0° C. to about 4° C. In the context of the method according to the invention, the collagen solution and buffer solution are brought to the desired temperature, for example to room temperature, i.e., in particular to 20° C. or more, or to body temperature, i.e., in particular about 30° C., but not to denaturing temperatures of more than 37° C., only shortly prior to use thereof for preparing the collagen biomatrix.

In particular, the collagen solution and buffer solution are brought to the desired temperature simultaneously, and in particular only immediately prior to mixing and dispensing according to the invention. This may be effected by brief storage in a heating cabinet, or optionally by warming in the hand. The invention seeks in particular to avoid any temperature of the collagen composition above 37° C.

According to the invention, the solutions at the desired temperature are mixed only at the time of or for application into the cavity or other site of application in order to thus in the process create the collagen composition that begins to gel instantaneously upon application, and gels, i.e. cures, in the site of application.

Both collagen solution and buffer solution are present in liquid form prior to use or application. Their low viscosity advantageously allows the immediate mixing thereof without additional, denaturing measures such as heating. The dynamic viscosity of the buffer solution is preferably within the range of that of water or of freely moving aqueous solutions, i.e., approximately 1 to 5 mPa.s. The dynamic viscosity of the concentrated collagen solution is preferably of the order of magnitude of approximately 10¹ to 10⁵ mPa.s.

The collagen solution and buffer solution are initially provided separately from each other, preferably in a multi-chamber syringe. They are brought to the desired temperature in particular separately from each other in the syringe and mixed immediately while being dispensed from the syringe. In particular, it is provided that combining and mixing the collagen solution and buffer solution is effected by expressing the solutions from the chambers of the syringe and by combining the solution flows within the syringe in a mixing apparatus associated with the syringe, the freshly prepared collagen composition flowing from the outlet of the mixing device in the process.

The invention facilitates the preparation of a collagen-containing biomatrix or collagen implant having a high ratio of non-denatured, cross-linkable collagen of native structure. It has been shown that cartilage cells proliferate particularly well in a collagen biomatrix, and have a high rate of collagen synthesis of their own when the biomatrix contains a high ratio of native collagen. The invention thus avoids any collagen-denaturing measures.

In some embodiments, the invention provides that the concentrated collagen solution provided in the context of the method according to the invention is obtained directly and without denaturing steps from collagen-containing tissue. In some embodiments, the collagen-containing tissue consists of prepared rat tail tendons. The collagen may, for example, be obtained therefrom by means of acid, such as acetic-acid, extraction. The concentrated collagen solution used may comprise_an acidic collagen solution having a collagen content (collagen concentration) greater than 8 mg/ml, alternatively approximately 9 mg/ml or greater, or alternatively up to about 16 mg/ml. The concentrated collagen solution is acidic in order to maintain the viscosity thereof. The pH of the concentrated collagen solution (based on 21° C.) is 6 or less, in some embodiments_within the range of pH 5 to pH 3.5.

In one particular embodiment, the concentrated collagen solution does not contain any further additives or excipients, such as cells; cell components; growth factors, such as cytokines; immunostimulants; antibiotics; or stabilizers, such as polysaccharides. In an alternative embodiment, at least one such additive or excipient is present in the collagen solution.

In order to obtain the gelling collagen composition, the concentrated acidic collagen solution is mixed with a neutralizing buffer. The neutralizing buffer is, in the simplest case, a known buffer salt solution, whereby the pH of the collagen composition is brought to a neutral range, in particular of pH 7.0 to pH 7.5 (based on a temperature of 21° C.). In some embodiments, a HEPES-buffered saline with a pH of 8.3 is used, which can be produced in a manner known. According to some embodiments of the invention, the buffer solution also serves to dilute the concentrated collagen solution for the purpose of achieving the final concentration in the gelling collagen composition. The buffer composition may, in some embodiments, be concentrated at least two-fold (at 1+1) and preferably concentrated maximally ten-fold (at 9+1), depending on the desired mixing ratio with the concentrated collagen solution.

In one particular embodiment, the buffer solution does not contain any further additives or excipients, such as cells; cell components; growth factors, such as cytokines;

immunostimulants; or stabilizers, such as polysaccharides. In an alternative embodiment, the buffer solution contains cells and optionally at least one further additive or excipient that form, when mixed according to the invention with the concentrated collagen solution, a cell-containing gelling collagen composition that cures to form a cell-containing collagen transplant. In a particular variant thereof, the cells are chondrocytes, in particular autologous cells and stem cells.

According to some aspects of the invention it is preferable to mix collagen solution and buffer solution at a volume ratio of 1+1 to 9+1 (collagen to buffer solution). In some particular embodiments, mixing ratios of 4+1 may be used, i.e., four parts of collagen solution to one part of buffer solution. Accordingly, the collagen content in the prepared collagen composition may, in some embodiments, be at least 6 mg/ml or more, in particular 6 to 12 mg/ml. In a particular embodiment thereof, the collagen content in the collagen composition is about 8 mg/ml.

In another aspect of the invention, a multi-chamber syringe may be used. This syringe is in a particular embodiment a known disposable syringe. In one possible variant, the syringe is equipped with a known static mixer as the mixing device. A person skilled in the art would also be familiar with other integral mixing devices that facilitate intermixing of separate solutions during application. In alternative embodiments, such other arrangements are also the subject matter of the invention.

If collagen solution and buffer solution are provided in a multi-chamber syringe, the device has chamber volumes of in particular about 0.5 to about 5 ml per chamber. Bringing the solutions to the desired temperature according to the invention takes place in this case inside the syringe, as does the mixing of the collagen solution and buffer solution when the solution is being dispensed from the syringe. In some embodiments, the mixing device of the syringe is a known static mixer. A person skilled in the art would be familiar with alternative mixing devices that can be used in conjunction with syringes. The process of dispensing may, for example, take approximately 1 to 60 seconds, depending on the syringe volume and the dispensing rate. According to the invention, the mixing time for each of the infinitesimal volume proportions flowing from the two chambers is, for example, about 0.5 to about 2 seconds.

The nascent composition begins to gel instantaneously, in particular within 10 seconds after mixing, or alternatively within 5 seconds. The gelling composition is still liquid and still flowable. The gelling process is complete when the collagen composition cures to form a solid biomatrix. The process of curing to form a biomatrix is complete preferably within 2 to 4 minutes. For example, in this manner, curing of the initially still flowable collagen composition applied into the cavity of the cartilage defect takes place only once the collagen composition reaches the cavity, such that a cartilage implant will form there that is fitted directly to the cavity.

Because the method according to the invention makes it possible to transport the prepared collagen composition to the application site, for example the cavity, while still in liquid form, smaller access ports are required for the site. This aids surgery performed through arthroscopic or endoscopic access ports. The previously necessary transport of a known, already cured ready-made cartilage implant through an endoscopic access port to the site is eliminated. The invention facilitates improved arthroscopic or minimally invasive surgery and thus reduces the trauma caused by the surgery.

In this context, another aspect of the invention also provides a ready-filled syringe, in particular in the form of a disposable syringe as a kit, the syringe incorporating at least two separate chambers that open into a dispensing device associated with the syringe, through which the content of each chamber can be dispensed, in some embodiments simultaneously. According to the invention the syringe is characterized in that at least one first chamber is filled with the liquid concentrated collagen solution and at least one second chamber separated therefrom is filled with the buffer solution. Furthermore, additional such chambers may be provided on the syringe, which may contain additives or excipients. The syringe that is filled at least with inventive collagen solution and buffer solution constitutes a kit that can be used for preparing an instantaneously gelling collagen composition.

In addition, the invention is not limited to the use of the method in a filled multi-chamber syringe. Other embodiments are conceivable that enable separate storage of concentrated collagen solution and buffer solution, and subsequent instantaneous mixing of these solutions for preparation of the gelling collagen composition. An alternative embodiment is in particular a single-use multi-chamber mixing capsule that can be used in conjunction with a known capsule mixer.

A further subject matter of the invention is the use of the filled syringe according to the invention for use in medical treatment. One particular application is the prophylactic or therapeutic treatment of cartilage defects in the human or animal body. These cartilage defects occur especially in joints. The use is not limited to articular cartilage defects, however. In fact, the invention can be used for treating further cartilage defects and other tissue defects in the human or animal body.

Other medical applications of the method according to the invention or of the filled syringe according to the invention include the treatment of tissue loss in the nucleus after disc herniation, and treatment of soft tissue defects in the skin, of bone and tendon defects and repair of gingival defects and other applications in dental and maxillofacial surgery, as well as applications in plastic surgery, in particular where defects or cavities need to be filled.

A further subject matter of the invention is the non-medical use of the method according to the invention and of the syringe filled according to the invention for preparing a collagen-containing biomatrix for tissue cultures. In one particular embodiment, a biomatrix for cell cultures can be prepared instantaneously by using the methods according to the invention. This is particularly suited for preparing sandwich cultures in which cells or tissue are to be coated with a collagen-containing biomatrix for the purpose of embedding the cells or tissue therein. In addition to further medical applications, there are a large number of non-medical applications in which a simple and reliable preparation of an instantaneously gelling collagen gel is needed.

The invention is described in further detail by the figures and the embodiments below without, however, being limited thereby.

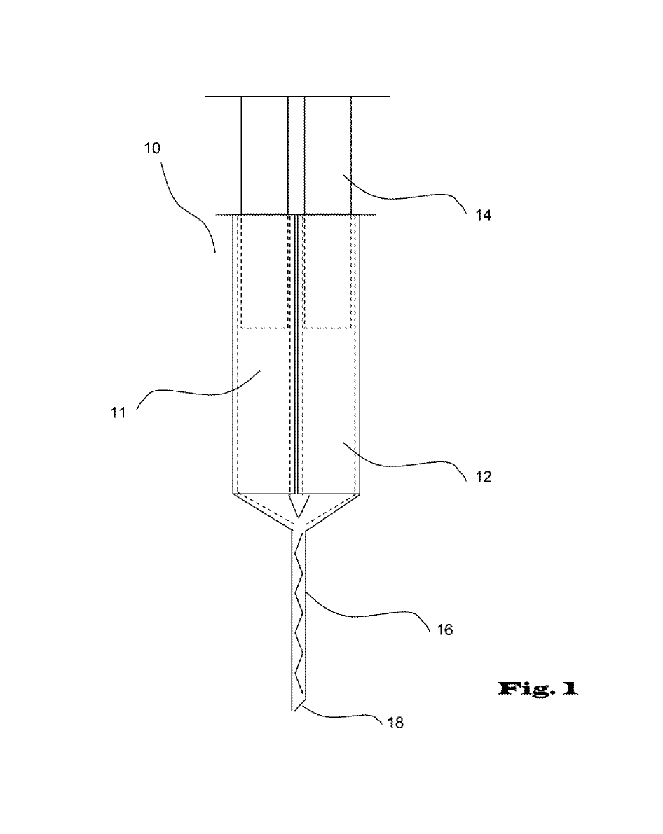

FIG. 1 shows a schematic view of an embodiment of the filled syringe according to the invention as a means for carrying out the method according to the invention.

In the embodiment shown, two separate parallel cylindrical chambers (11, 12) for accommodating the collagen solution on the one hand and the buffer solution on the other hand are formed in an integral container. Both chambers open at the front into a common outlet channel (18) comprising a static mixer (16) having a mixing baffle. Coupled syringe plungers (14) are provided for emptying the chambers (11, 12) for application and preparation of the collagen composition. These syringe plungers form the rearward delimitation of the volume of the chambers, where the content can be simultaneously expressed from both chambers (11, 12) in a known manner by applying pressure to the plunger (14). Depending on the ratio of the volume of the chambers (11, 12) predetermined by the design thereof, a homogenous mixing of the liquid contained in the chambers (11, 12) takes place in this volume ratio during the process of expression. In the illustrated embodiment, the volume ratio of the chambers (11, 12) is 1:1.

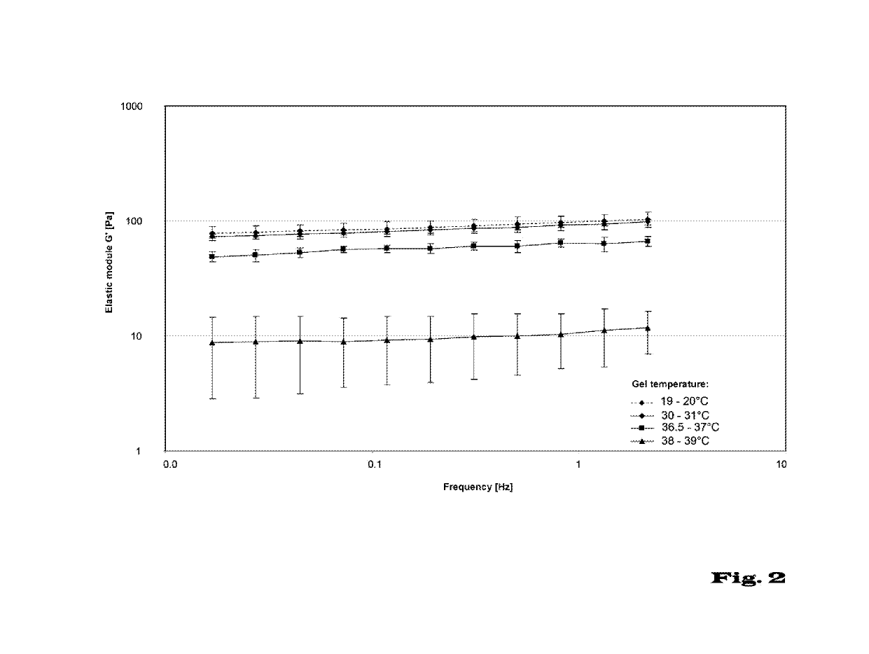

FIG. 2 shows the results of rheological studies on collagen gels: elastic modulus as a function of test frequency (mean values and standard deviations) in two-chamber syringes brought to different desired temperatures.

### EXAMPLE 1

### Producing an Instantaneously Gelling Collagen Composition

To prepare a concentrated collagen solution, rat tails are stored at approximately minus 20° C. and then superficially disinfected for a few minutes in an approximately 70% solution of alcohol. The skin is removed and the individual collagen fibers are dissolved out. The collagen fibers are again superficially disinfected in alcohol, then washed with PBS, subsequently transferred into an approximately 0.1% (0.5 mol/l) acetic acid solution and incubated therein. The collagen fibers are stirred in the acetic acid solution for a period of at least 7 days in the cold (at about 0 to 4° C.). After separating the undissolved collagen parts at the end of the incubation period, the collagen is filtered and precipitated. The precipitate is rinsed with buffer solution, frozen and then freeze-dried. The freeze-dried collagen is absorbed in 0.1% acetic acid as specified, such that a collagen content of 9 to 16 mg/ml is obtained. The pH of the concentrated collagen solution is approximately 4.0.

For mixing four parts of collagen solution with one part of neutralizing buffer solution (4+1), a five-fold concentrated buffer solution is prepared. In particular, a solution of 35.6 g NaCl in 937.5 ml ultrapure water with 62.5 ml of 3 mol/L HEPES solution is prepared as the five-fold concentrated buffer solution. The buffer solution is adjusted to pH 8.3 with NaOH prior to use.

The collagen solution and buffer solution are each filled into separate chambers of a multi-chamber syringe having a chamber volume ratio of 1:4, and stored until further use in the cold, at about −15° C. or colder.

For preparing a collagen-containing biomatrix, the multi-chamber syringe is briefly placed in a heating cabinet or water bath, whereby the buffer solution and collagen solution are heated to a temperature of about 30° C. For mixing the two solutions, same are expressed by the coupled syringe plungers from of the multi-chamber syringe. In the process, both solutions are passed through the mixing device connected to the chambers. The mixed, instantaneously gelling collagen composition flows from the syringe. It is filled into a casting mold while still in liquid form. After dispensing, the collagen composition completely fills the casting mold and within a few minutes cures to form a solid, collagen-containing biomatrix. At a collagen content of approximately 8 mg/ml of the collagen composition created by the mixing and a temperature of approximately 30° C., the collagen composition fully cures within 2 minutes.

### EXAMPLE 2

### Gelling of a Collagen Composition

The collagen compositions that can be prepared according to Example 1 are subjected to rheological analyses. Collagen solution (10 mg/ml) and buffer solution as per Example 1 are filled into dual chamber syringes (for example from Medmix Systems, Switzerland) (collagen chamber: 4 parts collagen solution, 2 ml; buffer chamber: 1 part five-fold concentrated buffer, 0.5 ml) and frozen.

The gentle thawing at room temperature (20.5° C.) for one hour is followed by ten minutes of bringing the syringes to the desired temperature in a water bath at different temperatures ranging from 20 to 40° C. (Table 1).

TABLE 1

| Desired temperature [° C.] | Number of syringes |
|---|---|
| (Actual temperature range [° C.]) | examined |
| 20 (19-20) | 3 |
| 30 (30-31) | 4 |
| 37 (36.5-37) | 4 |
| 38 (38-39) | 3 |
| 40 (40-41) | 4 |

For mixing the two components after bringing them to the desired temperature, the closure of the syringe is replaced with a mixing adapter and the content is carefully dispensed into a well of a 12-well tissue culture plate, the first two exiting drops being discarded.

Complete gelling of the collagen composition (8 mg/ml of collagen), i.e., curing to form a solid gel, took place within 15 min at 20.5° C. Consistency was then visually assessed (Table 2).

Subsequently, the elastic moduli of the gels are determined outside the wells by using frequency method (Bohlin CVO R 150, from Malvern Instruments GmbH, Germany) (FIG. 2).

TABLE 2

| Desired temperature [° C.] | Consistency of the |
|---|---|
| (Actual temperature range [° C.]) | gels after 15 min |
| 20 (19-20) | solid |
| 30 (30-31) | solid |
| 37 (36.5-37) | solid |
| 38 (38-39) | semi-solid |
| 40 (40-41) | liquid |

FIG. 2 shows that the highest determined average values of the elastic modulus are within a temperature range of 20 to 30° C. While the curve of the collagen warmed to 37° C. is somewhat lower, these gels also have a solid consistency on visual examination. When heated to 38° C., the gels were only semi-solid and had very low values with large fluctuations in the oscillation measurement. At a temperature of 40° C., the collagen no longer gels.

1. A method for preparing an instantaneously gelling collagen gel from a liquid concentrated collagen solution and a liquid buffer solution, comprising the following steps:
providing a separate collagen solution and buffer solution,
bringing the collagen solution and buffer solution each to a temperature of 20° C. to 37° C.,
mixing the collagen solution at the said temperature and buffer solution at the said temperature with each other, whereby a gellable collagen composition is obtained, which is capable of gelling instantaneously.

2. The method according to claim 1, wherein the collagen solution has a concentration greater than 8 mg/ml.

3. The method according to claim 2, wherein the pH of the collagen solution (based on a temperature of 21° C.) is 6 or less.

4. The method according to claim 1, wherein the buffer solution is a neutralizing buffer solution.

5. The method according to claim 1, wherein the collagen solution and buffer solution are mixed at a ratio of 1:1 to 9:1.

6. The method according to claim 1, wherein the prepared collagen composition has a collagen concentration of 6 mg/ml or greater.

7. The method according to claim 1, wherein mixing is completed within a maximum of 5 seconds.

8. The method according to claim 1, wherein the gelling of the prepared collagen composition begins within 10 seconds.

9. The method according to claim 1, wherein the collagen solution cures to form a solid gel within 2 to 4 minutes.

10. The method according to claim 1, wherein collagen solution and buffer solution are provided separately from one another in a multi-chamber syringe.

11. The method according to claim 10, wherein bringing the collagen solution and the buffer solution to the desired temperature takes place in the syringe.

12. The method according to claim 10 or 11, wherein combining and mixing of collagen solution and buffer solution is effected by expressing the solutions from the chambers of the syringe and combining the solution flows in a mixing device associated with the syringe, whereby the prepared collagen composition flows from the mixing device.

13. A filled syringe, containing at least two separate chambers that open into a mixing device associated with the syringe, through which the content of each chamber can be dispensed simultaneously, characterized in that at least one first chamber is filled with the liquid collagen solution at a temperature of 20° C. to 37° C. and at least one second chamber separated therefrom is filled with the buffer solution at a temperature of 20° C. to 37° C.

14. The syringe according to claim 13, wherein the mixing device is a static mixer.

15. A method for prophylactic or therapeutic treatment of a cartilage defect in a joint or organ of a human or animal body, comprising
providing a separate collagen solution and buffer solution,
bringing the collagen solution and buffer solution each to a temperature of 20° C. to 37° C.,
mixing the collagen solution at the desired temperature and buffer solution at the desired temperature with each other, whereby a gellable collagen composition is obtained, which is capable of gelling instantaneously,
placing the liquid gelling collagen composition into a cavity of the cartilage defect in the human or animal body.

16. The method according to claim 15, wherein the collagen solution has a concentration greater than 8 mg/ml.

17. The method according to claim 16, wherein the pH of the collagen solution (based on a temperature of 21° C.) is 6 or less.

18. The method according to claim 15, wherein the buffer solution is a neutralizing buffer solution.

19. The method according to claim 15, wherein the collagen solution and buffer solution are mixed at a ratio of 1:1 to 9:1.

20. The method according to claim 15, wherein the prepared collagen composition has a collagen concentration of 6 mg/ml or greater.
